SEQUENCE LISTING (4 sequences)

SEQ ID NO: 1 - DNA, 22 bp, Artificial Sequence
  16S conserved region
aaactcaaaggaattgacgggg

SEQ ID NO: 2 - DNA, 22 bp, Artificial Sequence
  16S primer
ccccgtcaattcctttgagttt

SEQ ID NO: 3 - DNA, 21 bp, Artificial Sequence
  Surface Strand, 5′ modification biotin
gaatcggtcacagtacaaccg

SEQ ID NO: 4 - DNA, 45 bp, Artificial Sequence
  capture adapter
ccccgtcaattcctttgagttttttggttgtactgtgaccgattc

(19) United States
(12) Patent Application Publication
(10) Pub. No.: US 2024/0301513 A1
(43) Pub. Date: Sep. 12, 2024

(54) SYSTEM AND METHOD FOR PERFORMING A MICROBIOME-ANALYSIS

(71) Applicant: MBIOMICS GMBH, Neuried (DE)

(72) Inventors: Johannes Benedikt WOEHRSTEIN, Hohenbachern (DE); Heinrich Ernst Paul GRABMAYR, München (DE)

(21) Appl. No.: 18/569,185

(22) PCT Filed: Jun. 14, 2021
(86) PCT No.: PCT/EP2021/065995

§ 371 Date: Dec. 11, 2023

(51) Int. Cl. C12Q 1/689; C12Q 1/6816; G01N 33/53; G01N 33/569; G01N 33/58

(52) U.S. Cl. CPC C12Q 1/689; C12Q 1/6816; G01N 33/5308; G01N 33/56911; G01N 33/582

(57) ABSTRACT

The present invention relates to a method for performing a microbiome-analysis, comprising the steps: a) providing a primary sample derived from a microbiomic sample, the primary sample comprising a first target species at a first concentration and a second target species at a second concentration and different from the first target species, wherein the first target species is indicative of a first element of a microbiome of the microbiomic sample and the second target species is indicative of a second element of the microbiome, and wherein the second concentration is higher than the first concentration; b) providing two or more secondary samples derived from the primary sample, the two or more secondary samples including at least a highest dilution secondary sample, a lowest dilution secondary sample and optionally one or more intermediate dilution secondary samples, wherein the at least two secondary samples have different dilutions, and wherein the lowest dilution secondary sample may be diluted or undiluted primary sample; c) providing a first fluorescent label that is configured to directly or indirectly bind specifically to the first target species to the lowest dilution secondary sample under conditions allowing for labeling the first target species with the first fluorescent label, and providing a second fluorescent label that is configured to directly or indirectly bind specifically to the second target species to the highest dilution secondary sample, but not to the lowest dilution secondary sample, under conditions allowing for labeling the second target species with the second fluorescent label; d) analyzing the first and second target species according to the steps; d1) imaging the secondary samples with fluorescence microscopy and counting the fluorescent first and second labels in the corresponding secondary samples; d2) calculating the ratio of the first target species to the second target species from the results of step d1) and the dilutions of the corresponding secondary samples.

The present invention relates to the field of microbiome analysis and particularly to systems and methods for performing a microbiome analysis. Herein, microbiome means the community of microorganisms and viruses inhabiting a common environment. Microbiomes typically comprise a large number of elements, which can be present in vastly different population sizes. The elements can be described at different taxonomic levels such as belonging to bacteria, archea, fungi, viruses, and eukaryotes, but also down to the species or strain level. These elements are often called operational taxonomic units (OTU). A typical example of a microbiome is the community of microorganisms and viruses inhabiting the intestine of a human.

With the field of microbiome analysis maturing, routine monitoring of the microbiome moves into focus. A technology to support that must be affordable, quick and simple to perform, and quantitative. By microbiome analysis, we understand the analysis of the composition of all or some of the elements of the microbiome, in terms of their population size. The analysis may include relative and/or absolute population sizes. The analyzed elements and the taxonomic level of their analysis/description depends on the specific question of interest. For example, a microbiome-analysis may be carried out on the taxonomic level of kingdoms. For example, a microbiome-analysis may analyze the number of bacteria, absolute or relative to other elements such as fungi etc., without distinguishing the lower taxonomic levels, e.g. individual bacteria species. A microbiome-analysis may also aim at analyzing at least some of the individual bacteria species, e.g. E. coli and C. difficile.

More and more, research shows the impact of especially the gastrointestinal microbiome on health (auto-immunity, cancer resistance, gut-brain axis) (see Zheng et al., Cell Research 2020, https://doi.org/10.1038/s41422-020-0332-7; Woelk et al, Cancer Imm 2021, https://doi.org/10.1126/science.abg2904; and Margolis et al, Gastroenterology 2021, https://doi.org/10.1053/j.gastro.2020.10.066). While the gastrointestinal microbiome has been shown to be variable between individuals, it is more stable within an individual over time (see Mehta et al., Nat Microbiol. 2018, https://doi.org/10.1038/s41564-017-0096-0). Subtle changes of the composition of the microbiome over time can allow insights into the status of the host organism. The composition of the microbiome may also be a proxy for the probability of adverse reactions to medical treatments.

In the future, diagnostic applications will require technologies that are applied repetitively before, during, and after a treatment, and are sensitive to change in the composition of the microbiome.

Today, microbiomes are analyzed either via the analysis of variable regions in the 16S and/or 23S ribosomal subunits (for archea and bacteria) and/or the corresponding analysis of the internal transcribed spacer (ITS, fungi) and/or 18S and/or 28S ribosomal subunits (eukaryotes). These sequences are specific to a taxonomic level, often down to the level of biological species. These sequences can be analyzed using different technologies, typically qPCR or sequencing.

The alternative is called shotgun metagenomic sequencing and is based on sequencing the total extracted DNA of the microbiome, and bioinformatically decomposing it into the known genomes of the potential constituents, i.e. elements, of the microbiome. Its benefits over 16S analysis are strain-level identification, high sensitivity, and the possibility to use the genetic information for further analysis. However, because of the highly complex reconstruction of the microbiome composition, it is not ideal for quantification.

Another method is quantitative microbiome profiling. It combines sequencing with other methods, e.g. flow cytometry, to relate sequencing results to cell count and thereby increase quantification.

These technologies have inherent noise sources like reverse transcription and enzymatic amplification, which results in rather high variability:

“16S qPCR estimates have been reported to have a high technical noise, with a coefficient of variation (CV) ranging from 11 to 75%” (Li et al. Microbiome (2019) 7:118 https://doi.org/10.1186/s40168-019-0729-z).

16S amplicon sequencing has a CV ranging around 10-175% (Li et al. Nat Commun (2020) https://doi.org/10.1038/s41467-020-16224-6).

Flow cytometry QMP, which is an example for quantitative microbiome profiling, has a CV of 2-50% (see Galazzo et al., Front. Cell. Infect. Microbio. (2020) https://doi.org/10.3389/fcimb.2020.00403, based on table S3).

Other drawbacks include the relatively low number of simultaneously analyzed elements, and the throughput of samples to analyze.

Initial measurements with synthetic DNA-oligomers as targets in J B Woehrstein et al. showed a mean error below 4%. Initial tests on 16S rRNA suggest errors of similar magnitude. The technology is also known as “multiplexing of fluorescent nanoparticles”.

In short, multiplexing of fluorescent DNA-nanoparticles allows for detection and quantification of target nucleic acids on the single molecule level. The target nucleic acids are immobilized on a surface. One or more fluorescent DNA-nanoparticles are hybridized with the immobilized target nucleic acids. Different types of target nucleic acids are labeled with different tags, i.e. with different types of fluorescent DNA-nanoparticles or different combinations of fluorescent DNA-nanoparticles, wherein the different tags are distinguishable in fluorescence microscopy. The target nucleic acids are quantified by imaging the fluorescent DNA-nanoparticles with fluorescence microscopy. The fluorescent DNA-nanoparticles are identified by de-multiplexing and counted.

Multiplexing of fluorescent nanoparticles stands out among other detection techniques due to its high accuracy in combination with a relatively large number of targets that may simultaneously be analyzed, short analysis times, and the type of resulting data which may directly be analyzed.

However, a problem with analyzing microbiomic samples is that the microbiomic components, i.e. elements, occur in a wide range of concentrations. For example, Bacteroides vulgatus, Bacteroides uniformis, and Alistipes putredinis often occur at a population size of 1-10% in human stool samples, while Bacteroides fragilis and Bacteroides coprophilus rather occur in the order of magnitude of 0.01%-0.1%, and Abiotrophia defective, Acidaminococcus fermentans occur in the range of 0.00001%-0.0001% (Kraal et al, Plos One 2014 https://doi.org/10.1371/journal.pone.0097279 (Supplementary Figure S2). So far, the analysis of microbiomic samples by multiplexing of fluorescent nanoparticles is limited to certain ranges of concentrations. Typical ranges are three to four orders of magnitude, i.e. the ratio of the highest to the lowest detectable concentration is 1000:1 to 10.000:1; depending on sample noise sources and imaging area.

Accordingly, it is an object of the present invention to provide methods and systems for microbiome analysis that enable quantification of different target species in a microbiomic sample, and particularly quantification of different target species over a wide range of concentrations of the target species, wherein the different target species are indicative of elements of the microbiome of the microbiomic sample. Advantageously, the methods and systems have short analysis times (as compared to known techniques), provide data that may directly be analyzed and have a high precision (as compared to known techniques).

The present invention increases the dynamic range of the method “multiplexed in vitro nucleic acid detection assay” and applies it to the analysis of a microbiomic sample. “Dynamic range”, a detailed explanation of which is given below, refers to the ratio of the highest to the lowest values a certain quantity can assume. It can be expressed as a ratio, or as the logarithm of the ratio (then usually in Decibel (dB) with base 10 or bits with base 2). In camera technology, the field the present invention is most related to, the plain ratio is preferred, and thus used here as well.

In microbiomic samples, the expected concentrations of the target elements to be analyzed, e.g. specific bacteria, etc., are usually known to a certain degree. This transfers to the target species that are indicative of the target elements. For example, in the case of a specific type of bacteria with an expected concentration the target species may be the respective 16S-rRNA with a proportional concentration. The proportionality factor and its certainty can be established in separate measurements as known in the art beforehand, or accessed from databases (e.g. the Ribosomal RNA Operon Copy Number Database, rrnDB, https://rrndb.umms.med.umich.edu/or AmpliCopyRighter, https://github.com/fangly/AmpliCopyRighter). The increase of the dynamic range is achieved by including several dilutions of a sample (which may also include non-diluted sample), wherein for each dilution the target species to be analyzed by multiplexing of fluorescent nanoparticles are selected according to the concentrations of target species and a dilution factor of the dilution and preferably the dynamic range of the system used for analysis. The present invention uses a more direct method, namely a multiplexed in vitro nucleic acid detection assay to detect and count single 16S rRNA molecules or other target species, without the introduction of the technical noise sources of reverse transcription and enzymatic amplification. The multiplexed in vitro nucleic acid detection assay with fluorescent DNA-nanoparticles is known, e.g., from J B Woehrstein et al., Science Adv (2017) https://doi.org/10.1126/sciadv.1602128, from WO2016140727 and from EP3472351.

In one aspect, the present invention relates to a first method for performing a microbiome-analysis, comprising the steps:
a) providing a primary sample derived from a microbiomic sample, the primary sample comprising a first target species at a first concentration and a second target species at a second concentration and different from the first target species, wherein the first target species is indicative of a first element of a microbiome of the microbiomic sample and the second target species is indicative of a second element of the microbiome, and wherein the second concentration is higher than the first concentration;
b) providing two or more secondary samples derived from the primary sample, the two or more secondary samples including at least a highest dilution secondary sample, a lowest dilution secondary sample and optionally one or more intermediate dilution secondary samples, wherein the at least two secondary samples have different dilutions, and wherein the lowest dilution secondary sample may be diluted or undiluted primary sample;
c) providing a first fluorescent label that is configured to directly or indirectly bind specifically to the first target species to the lowest dilution secondary sample under conditions allowing for labeling the first target species with the first fluorescent label, and providing a second fluorescent label that is configured to directly or indirectly bind specifically to the second target species to the highest dilution secondary sample, but not to the lowest dilution secondary sample, under conditions allowing for labeling the second target species with the second fluorescent label;
d) analyzing the first and second target species according to the steps:
   d1) imaging the secondary samples with fluorescence microscopy and counting the fluorescent first and second labels in the corresponding secondary samples;
   d2) calculating the ratio of the first target species to the second target species from the results of step d1) and the dilutions of the corresponding secondary samples.

Optionally, the first fluorescent label is not provided to the highest dilution secondary sample under conditions allowing for labeling the first target species with the first fluorescent label.

The microbiomic sample may be, as already mentioned above, a stool sample or any derivative thereof.

As already mentioned above, herein the term “target species” refers to a type of target, e.g. a type of target molecule, such as a type of target DNA or target RNA, e.g. a type of 16S-rRNA (e.g. the 16S-rRNA of the bacterium Escherichia coli), a type of 18S-rRNA (e.g. the 18S-rRNA of the yeast Saccharomyces boulardii). It is not to be confused with the biological species.

The primary sample is a sample that comprises the target species in conditions that are suitable for the method described above. Particularly, in the primary sample the target species may have been extracted from the target elements of the microbiomic sample. For example, the primary sample may comprise 16S-rRNA extracted from and indicative of corresponding target elements of the microbiomic sample (e.g. a stool sample, a saliva sample, skin swab or also non-human microbiome samples like plant microbiome, marine microbiome samples, sewer samples, or cultured microbiomes). The sample may be purified to contain only selected classes of molecules, e.g. nucleic acids, whole RNA or ribosomal RNA.

Highest dilution secondary sample means the secondary sample with the highest dilution factor, i.e. the secondary sample that has been diluted the most, and consequently has the lowest concentrations of target species.

The lowest dilution secondary sample is the secondary sample with the lowest dilution factor, i.e. the secondary sample that has been diluted the least, and consequently has the highest concentrations of target species. In the case of the lowest dilution secondary sample being undiluted primary sample, the concentrations of target species are identical to the concentrations of target species in the primary sample. The lowest dilution secondary sample being undiluted primary sample also includes the case that primary sample that has been diluted and re-concentrated to the concentrations of the primary sample.

The method is particularly useful if a ratio of the second concentration to the first concentration is sufficiently close to or beyond the dynamic range of the measurement system, for example at least one order of magnitude below the dynamic range of the used measurement system. For example, if the dynamic range of the measurement system is 10³:1, the method is particularly useful for the ratio of the second concentration to the first concentration being 100:1 or larger (such as 10³:1, 10⁵:1, 10⁷:1, 10⁸:1). If the dynamic range of the measurement system is 10⁴:1, the method is particularly useful for the ratio of the second concentration to the first concentration being 10³:1 or larger, and so on. The method enables quantification of the first and second target species even for the case that the range of concentrations of the first and second target species in the sample is close to and/or beyond the dynamic range of the measurement system. Even if the concentrations of the first and second target species in the sample are not close to and/or not beyond the dynamic range of the measurement system, the method may be advantageous. In this case, both target species can be measured simultaneously at multiple dilutions to yield a more precise measurement. If the dynamic range of the measurement system is 10⁴:1 and the ratio of first concentration to the second concentration is 10²:1, the sample can be measured at dilution 1 and 100, and the consistency of the results can be analyzed.

Specific binding takes place when two molecules are locked together in a mechanism with a well-defined affinity. It can occur via hybridization of complementary single strands of nucleic acids, in protein-ligand (such as e.g. biotin-streptavidin) interaction, as well as antibody-antigen interaction. Finally, it may also describe the interaction of chemical binding partners, such as NHS-ester-amine; maleimide-thiol, and in general bioconjugation reactions. The interaction forces are mainly Van der Waals bonds, hydrogen bonds, and ionic interactions, or covalent bonds in the case of bioconjugation. It can be used to immobilize one type of molecule from solution by providing an immobilized interaction partner. Specific binding contrasts to unspecific binding, which is based on the same type of forces, but not with a well-defined affinity. Therefore, repetitive trials of binding between the partners don't yield consistent results. The term “unspecific binding” is often used to describe the fact that a molecule sticks to something that isn't designed to act as an interaction partner.

Direct binding of a component A to a component B means, that component A binds to component B without any additional component in between. For example, a single stranded DNA A may directly bind to a complementary single stranded DNA B via hybridization. Indirect binding of a component A to a component B means, that component A binds to component B with an additional component in between. For example, a single stranded DNA A may indirectly bind to a single stranded DNA B via a single stranded DNA C, wherein a first portion of the single stranded DNA C is complementary to and hybridizes with a portion of the single stranded DNA A and a second portion of the single stranded DNA C is complementary to and hybridizes with a portion of the single stranded DNA B. Even if not specifically stated, binding is always meant to include direct and/or indirect binding.

“Not providing component X to a sample (under conditions for binding)” may include providing component X to a sample wherein very low binding that does not disturb the measurement occurs.

“Not providing component X to a sample (under conditions for binding)” may include providing component X to a sample wherein binding occurs to such a small extent (with such a small affinity), that an insignificant amount of component X remains in the sample after the last wash step in the protocol. In the end, the amount after washing is insignificant if it is detected at the level of the noise floor in the resulting data, independent of the initial amount of component X.

Resulting in a second method, step c) may comprise the steps:
c1) applying the secondary samples to corresponding coverslip regions, wherein the coverslip regions are modified according to the corresponding secondary sample and in the following way:
   c1.1) each coverslip region is passivated against unspecific binding of the first and second target species and optionally against unspecific binding of the fluorescent labels;
   c1.2) each coverslip region is covered with immobilization molecules bound, directly or indirectly, to the coverslip, wherein the immobilization molecules are configured to, directly or indirectly, bind the first and the second target species;
c2) optionally removing unbound individuals of the first and second target species from the coverslip regions;
c3) providing the first fluorescent label to the lowest dilution secondary sample under conditions allowing for labeling the first target species with the first fluorescent label, and providing the second fluorescent label to the highest dilution secondary sample but not to the lowest dilution secondary sample, under conditions allowing for labeling the second target species with the second fluorescent label;
c4) optionally removing unbound individuals of the first and second fluorescent labels from the coverslip regions.

The coverslip regions may be located on one and the same coverslip or on two or more coverslips. For example, each coverslip region may be located on another coverslip. In another embodiment, some of the coverslip regions may be located on one and the same coverslip while the remaining coverslip regions are each located on their own coverslip. Any combination is possible.

Step c1) is an immobilization step for binding the first and second target species to the coverslip regions. In step c1), the secondary samples are applied to corresponding coverslip regions. As the coverslip regions are passivated against unspecific binding (item c1.1) of the first and second target species to the coverslip regions, essentially only binding of the first and second target species to the immobilization molecules bound to the coverslip (item c1.2) may occur.

The order of steps c1), c2) and c3) may be any suitable order, as long as c2) is after c1). Preferably the order is: First c1), followed by c2), followed by c3). However, the order may also be: first c1), followed by c3), followed by c2). The order may also be: first c3), followed by c1), followed by c2). In the case of “first c3), followed by c1), followed by c2)”, a complex of the target species with the corresponding fluorescent label(s) is allowed to be formed prior to immobilization in step c2). The order of c1), c2) and c3) may be selected individually for each target species and each suitable combination of orders is contemplated. As mentioned above, c2) is an optional step.

Step c4) may be performed additionally or alternatively to step c2), and always after c3). If both c2) and c4) are performed after c3), step c2) may be one and the same step as step c4). The order of steps c1), c2), c3) and c4) may be any suitable order, as long as c4) is after c3) and c2) is after c1). Preferably the order is: First c1), followed by c3), followed by c4) and/or c2). However, the order may also be: first c1), followed by c2), followed by c3), followed by c4). The order may also be: first c3), followed by c1), followed by c2) and/or c4). In the case of “first c3), followed by c1), followed by c2) and/or c4)”, a complex of the target species with the corresponding fluorescent label(s) is allowed to be formed prior to immobilization in step c2). The order of c1), c2), c3) and c4) may be selected individually for each target species and each suitable combination of orders is contemplated. As mentioned above, c2) and c4) are optional step.

A general idea of the methods described so far is the following: The selection of the different target species that are to be analyzed in the individual secondary samples is achieved via the fluorescent labels. Both the first and second target species are provided to the secondary samples and optionally corresponding coverslip regions, and the fluorescent labels are selectively added to the secondary samples under binding conditions.

However, such selection may also be achieved by selective immobilization of different target species. Accordingly, the present invention also relates to a third method for performing a microbiome-analysis, comprising the steps:
a) providing a primary sample derived from a microbiomic sample, the primary sample comprising a first target species at a first concentration and a second target species at a second concentration and different from the first target species, wherein the first target species is indicative of a first element of a microbiome of the microbiomic sample and the second target species is indicative of a second element of the microbiome, and wherein the second concentration is higher than the first concentration;
b) providing two or more secondary samples derived from the primary sample, the two or more secondary samples including at least a highest dilution secondary sample, a lowest dilution secondary sample and optionally one or more intermediate dilution secondary samples, wherein the at least two secondary samples have a different dilution, and wherein the lowest dilution secondary sample may be diluted or undiluted primary sample;
c) providing a first fluorescent label that directly or indirectly binds specifically to the first target species to the lowest dilution secondary sample under conditions allowing for labeling the first target species with the first fluorescent label and providing a second fluorescent label that directly or indirectly binds specifically to the second target species to the highest dilution secondary sample under conditions allowing for labeling the second target species with the second fluorescent label; and
   wherein in the lowest dilution secondary sample the first target species is being immobilized for analysis and the second target species is not being immobilized for analysis, wherein in the highest dilution secondary sample the second target is immobilized for analysis and optionally the first target is not immobilized for analysis;
d) analyzing the first and second target species according to the steps:
   d1) imaging the secondary samples with fluorescence microscopy and counting the first and second fluorescent labels in each secondary sample;
   d2) calculating the ratio of the first target species to the second target species from the results of step d1) and the dilutions of the corresponding secondary samples.

Resulting in a fourth method, step c) of the third method may comprise the steps:
c1) applying the secondary samples to corresponding coverslip regions, wherein the coverslip regions are modified according to the corresponding secondary sample and in the following way:
   c1.1) each coverslip region is passivated against unspecific binding of the first and second target species and optionally against unspecific binding of the fluorescent labels;
   c1.2) the coverslip region corresponding to the lowest dilution secondary sample is covered with first immobilization molecules bound, directly or indirectly, to the coverslip, wherein the first immobilization molecules are configured to, directly or indirectly, specifically bind the first target species;
   c1.3) the coverslip region corresponding to the highest dilution secondary sample is covered with second immobilization molecules bound, directly or indirectly, to the coverslip, wherein the second immobilization molecules are configured to, directly or indirectly, specifically bind the second target species;
c2) optionally removing unbound individuals of the first and second target species from the coverslip regions;
c3) providing the first fluorescent label to the lowest dilution secondary sample under conditions allowing for labeling the first target species with the first fluorescent label, and providing the second fluorescent label to the highest dilution secondary sample under conditions allowing for labeling the second target species with the second fluorescent label and optionally not providing the second fluorescent label to the lowest dilution secondary sample under conditions allowing for labeling the second target species with the second fluorescent label
c4) optionally removing unbound individuals of the first and second fluorescent labels from the coverslip regions.

The definitions and explanations given above with respect to the first and second methods, particularly the order of steps c1), c2), c3) and c4), apply mutatis mutandis to the third and fourth methods.

Resulting in a fifth method, the fourth method may comprise the step of predetermining a first threshold dilution factor, and wherein step c1) of the fourth method comprises the following condition for modifying the coverslip regions:
c1.4) the coverslip regions corresponding to the intermediate dilution secondary samples are covered with first and/or second immobilization molecules bound, directly or indirectly, to the coverslip, wherein the coverslip regions corresponding to the secondary samples having a dilution factor greater than the first threshold dilution factor are not covered with first immobilization molecules.

The first threshold dilution factor, and also the other threshold dilution factors mentioned below, may be determined explicitly or implicitly. Explicitly determining the threshold dilution factor may be calculating the respective value. Implicitly determining the threshold dilution factor may be calculating a different value that may be recalculated to a threshold dilution factor and/or results in dividing the intermediate secondary samples in those for covering and those for non-covering.

Resulting in a sixth method, the fourth or fifth method may comprise the step of
predetermining a second threshold dilution factor, and wherein step c1) comprises the following condition for modifying the coverslip regions:
c1.5) the coverslip regions corresponding to the intermediate dilution secondary samples are covered with first and/or second immobilization molecules bound, directly or indirectly, to the coverslip, wherein the coverslip regions corresponding to the secondary samples having a dilution factor equal to or smaller than the second threshold dilution factor are not covered with second immobilization molecules,
optionally wherein the second threshold dilution factor is equal to the first threshold dilution factor.

According to another aspect of the present invention, a method may comprise the step of predetermining a third threshold dilution factor, and wherein the intermediate dilution secondary samples having a dilution factor greater than the third threshold dilution factor are provided with the second fluorescent label under conditions allowing for labeling the second target species with the second fluorescent label and the intermediate dilution secondary samples having a dilution factor equal to or lower than the third threshold dilution factor are not provided with the second fluorescent label under conditions allowing for labeling the second target species with the second fluorescent label; optionally wherein the third threshold dilution factor is equal to the first and/or the second threshold dilution factor(s).

According to another aspect of the present invention, a method may comprise the step of predetermining a fourth threshold dilution factor, and wherein the intermediate dilution secondary samples having a dilution factor equal to or lower than the fourth threshold dilution factor are provided with the first fluorescent label under conditions allowing for labeling the first target species with the first fluorescent label and optionally the intermediate dilution secondary samples having a dilution factor greater than the third threshold dilution factor are not provided with the first fluorescent label under conditions allowing for labeling the first target species with the first fluorescent label.

The fourth threshold dilution factor may be equal to the first, the second and/or the third threshold dilution factor(s).

The number of intermediate dilution secondary samples may be adapted to the expected concentrations and/or to the ratio of the expected concentrations. For example, the number of intermediate dilution secondary samples can be adapted such that each expected concentration lies in the linear measurement regime once. If the measurement system has a dynamic range of 10³:1 and the ratio of the expected first and second concentrations (second concentration/first concentration) is 10⁵, two secondary samples are needed, while if the ratio of the expected first and second concentrations is 10²:1, only one secondary sample is needed. In another example with a measurement system dynamic range of 10³:1, and where a first, second, and third species at a first, second and third concentration, respectively, with the ratio of first and second concentration (second concentration/first concentration) being 10⁶ and the ratio of first and third concentration (third concentration/first concentration) being 10³, shall be measured, three secondary samples will be used. In yet another example with the same three target species and concentrations but with a measurement system dynamic range of 10⁴:1, it is sufficient to only use two secondary samples.

The number of intermediate dilution secondary samples can be adapted such that the uncertainty of expected concentrations can be taken into account. If the measurement system has a dynamic range of 10²:1 and the ratio of the expected first and second concentrations (second concentration/first concentration) is 10³, but both the first and second concentrations are expected to vary one order of magnitude up or down, the total concentration range to be assayed is 10⁵, and therefore three coverslip regions and thus one intermediate coverslip region can be employed. With this, measuring both the first and second target species in the linear regime can be ascertained with a higher confidence. In a third example, the measurement accuracy can be increased by measuring one target at multiple dilutions. If the measurement system has a dynamic range of 10²:1 and the ratio of the expected first and second concentrations (second concentration/first concentration) is 10³, and each target should be assayed at two dilution steps, a total of at least three dilution samples (i.e. secondary samples) are needed, i.e. at least one intermediate dilution secondary sample is required.

Additionally or alternatively, the dilutions of the secondary samples may be adapted to the expected first and expected second concentrations and/or to the ratio of expected first and expected second concentrations. As compared to the embodiment with standard dilutions, that are used independently of the specific concentrations of the target species, the embodiment with adapted dilutions may have several advantages. The number of secondary samples may be reduced. This may not only save material but also time, particularly time for method step d).

Adapting the number and/or dilutions of the secondary samples as mentioned above may also contribute to verify the accuracy of the inventive methods. For example, it is preferable to always measure at least two to three secondary samples, i.e. dilutions, for each target species. From such number of measurements, it is possible to identify secondary samples with saturation conditions in step c1). Saturation conditions in step c1) means that the number of binding sites for the respective target species on a coverslip region was not sufficient for all individuals of the target species in the secondary sample to bind to the coverslip region.

The first target species may be prepared for analysis and analyzed in one or more intermediate dilution secondary samples.

Additionally or alternatively, the second target species may be prepared for analysis and analyzed in one or more intermediate dilution secondary samples.

The coverslip regions may be comprised in a fluid-manipulation device, preferably a microfluidic device, with each coverslip region at least partially defining a corresponding fluid reaction compartment, preferably a microfluidic reaction compartment, each reaction compartment comprising an inlet; and wherein the application of the secondary samples and/or the application of the DNA-nanostructures to the corresponding coverslip regions is performed via the respective inlets.

A fluid-manipulation device is a device for manipulating, i.e. processing one or more fluids. A fluid-manipulation device may be a flow chamber or a well chamber. The most prominent example is a microfluidic device. However, also devices with dimensions beyond the dimensions of microfluidic devices are contemplated.

The inlets may also serve as outlets, i.e. for extracting fluid from the reaction compartments. Alternatively or additionally, the reaction compartments may comprise separate outlets for extraction of fluid from the reaction compartments. All reaction compartments may have the same configuration of inlets and outlets, or some reaction compartments may have a combined inlet and outlet, while others have separate inlets and outlets.

The first fluorescent label and/or second fluorescent label may comprise at least one DNA-nanostructure with at least one fluorescent dye. Two cases may be distinguished, a “non-overlapping case” and an “overlapping case”. In a non-overlapping case in each secondary sample only one of the first and second target species is prepared for analysis, e.g. labeled.

In an overlapping case in at least one secondary sample both the first and the second target species are prepared for analysis, e.g. labeled, with the first and the second fluorescent labels, respectively. In the non-overlapping case, the second fluorescent label when bound to the second target species may or may not be distinguishable in fluorescence microscopy from the first label when bound to the first target species. In the overlapping case, the second fluorescent label when bound to the second target species must be distinguishable in fluorescence microscopy from the first label when bound to the first target species, at least in those secondary samples in which both target species are to be analyzed.

A person skilled in the art knows how to select and/or create suitable fluorescent labels, particularly, suitable fluorescent labels comprising at least one DNA-nanostructure. The necessary information may be found in literature, e.g. in Woehrstein et. al cited above, DE102012107719, WO2019149932, WO2012058638, WO2017100251, US2014031243, WO2016140727 and/or EP3472351.

The first target species may comprise a first nucleic acid, preferably a first 16S-rRNA, the first nucleic acid comprising a sequence portion S1 specific for the first nucleic acid, and wherein the first fluorescent label comprises a sequence portion S3 at least partially complementary to the sequence portion S1. This may enable specific binding of the first fluorescent label to the first target species.

The second target species may comprise a second nucleic acid, preferably a second 16S-rRNA, the second nucleic acid comprising a sequence portion S2 specific for the second nucleic acid, and wherein the second fluorescent label comprises a sequence portion S4 at least partially complementary to the sequence portion S2. This may enable specific binding of the second fluorescent label to the second target species.

The immobilization molecules may comprise oligonucleotides and the specific binding to corresponding target species may occur via hybridization of at least partially complementary sequence portions on the oligonucleotides and the corresponding target species.

In methods that include the first method and in which the first and second target species comprise nucleic acids, the immobilization molecules may comprise one type of oligonucleotides that is configured to bind both the first and the second target species. For example, if the first target species is a first mRNA with a poly-A-tail and the second target species is a second mRNA with a poly-A-tail, the oligonucleotide may comprise a corresponding poly-T-sequence that is configured to bind to the poly-A-tail, i.e. either one of the first and the second target species. 16S-rRNAs also have a conserved region in their nucleic acid sequences. If the first and second target species are 16S-rRNAs, they may comprise a sequence Seq-ID No 1 “AAACTCAAAGGAATTGACGGGG” (see Wang et al., Encyclopedia of Metagenomics 2013, DOI 10.1007/978-1-4614-6418-1_772-1, Table 2) the oligonucleotide may comprise a corresponding complementary sequence that is configured to bind the Seq-ID No 1, i.e. either one of the first and second target species. This may e.g. be Seq-ID No 2.

In methods in which the first and second target species comprise nucleic acids, the immobilization molecules may comprise a first oligonucleotide that is configured to bind the first target species but not the second target species and a second oligonucleotide that is configured to bind the second target species but not the first target species. This feature is not only suitable for methods including the first method but also for methods including the third method.

The inventive method may include a preparation of the primary sample from a microbiomic sample. For example, the microbiomic sample may be a stool sample, e.g. of a human or an animal. The method may include collecting such stool sample. The method may include purifying the first and the second target species from the microbiomic sample, e.g. the stool sample, thus providing the primary sample. Techniques are known in the art and routinely used. Preferably, a technique is used that conserves the ratio of the target species of interest. If this is not the case, an analysis may still make sense, e.g. if the ratios of target species in the purified sample, i.e. primary sample, are monitored for an individual, e.g. a person, to investigate, whether a treatment of this individual does affect the microbiome of this individual.

So far, the invention has been described with reference to two target species, the first and the second target species. Although this description does include embodiments in which more than two target species are analyzed, a description of the invention with reference to several target species is given in order to explain the full advantages of the invention.

In analogy to the first method, the present invention relates to a seventh method for performing a microbiome-analysis, comprising the steps:
A) providing a primary sample derived from a microbiomic sample, the primary sample comprising m target species at respective concentrations cᵢ,
   wherein each of the m target species is indicative of an element of a microbiome of the microbiomic sample;
B) providing u secondary samples derived from the primary sample, the u secondary samples including at least a highest dilution secondary sample, a lowest dilution secondary sample and optionally one or more intermediate dilution secondary samples,
   wherein the u secondary samples have different dilutions, and wherein the lowest dilution secondary sample may be diluted or undiluted primary sample;
C) selecting for each secondary sample
   C1) those one or more target species among the m target species that are to be prepared for analysis by providing corresponding fluorescent labels that are configured to directly or indirectly bind specifically to the target species under conditions allowing for labeling the one or more target species with the respective fluorescent labels, wherein the fluorescent labels corresponding to different target species are distinguishable in fluorescence microscopy, and
   C2) the corresponding fluorescent labels that are configured to directly or indirectly bind specifically to the one or more target species selected in step C1) under conditions allowing for labeling the one or more target species with the respective fluorescent labels;
D) providing the selected fluorescent labels to the corresponding secondary samples as selected in step C) under conditions allowing for labeling the respective target species with the selected fluorescent labels;
E) analyzing the m target species according to the steps:
   E1) imaging the secondary samples with fluorescence microscopy and counting the fluorescent labels in the corresponding secondary samples;
   E2) calculating the ratios of the m target species from the results of step E1) and the dilutions of the corresponding secondary samples.

In analogy to the third method, the present invention relates to an eighth method for performing a microbiome-analysis, comprising the steps:
A) providing a primary sample derived from a microbiomic sample, the primary sample comprising m target species at respective concentrations cᵢ,
   wherein each of the m target species is indicative of an element of a microbiome of the microbiomic sample;
B) providing u secondary samples derived from the primary sample, the u secondary samples including at least a highest dilution secondary sample, a lowest dilution secondary sample and optionally one or more intermediate dilution secondary samples,
   wherein the u secondary samples have different dilutions, and wherein the lowest dilution secondary sample may be diluted or undiluted primary sample;
C) selecting for each secondary sample
   C1) those one or more target species among the m target species that are to be prepared for analysis by immobilization, and
   C2) corresponding fluorescent labels that are configured to directly or indirectly bind specifically to the one or more target species selected in step C1) under conditions allowing for labeling the one or more target species with the respective fluorescent labels, wherein the fluorescent labels corresponding to different target species are distinguishable in fluorescence microscopy;
D) preparing the secondary samples by
   D1) immobilizing the target species according to the selection in step C1)
   D2) providing the selected fluorescent labels to the corresponding secondary samples as selected in step C) under conditions allowing for labeling the respective target species with the selected fluorescent labels;
E) analyzing the m target species according to the steps:
   E1) imaging the secondary samples with fluorescence microscopy and counting the fluorescent labels in the corresponding secondary samples;
   E2) calculating the ratios of the m target species from the results of step E1) and the dilutions of the corresponding secondary samples.

“Fluorescent labels are distinguishable in fluorescence microscopy” means that the fluorescent labels are distinguishable with an appropriate fluorescence microscopy system, the appropriate settings of this system, an appropriate image processing and an appropriate data analysis, all of which is known to a person skilled in the art. Obviously, there is a very large number of possible combinations of devices and fluorescent labels, which are routinely used in the technical field, such that specific feature combinations are neither necessary nor meaningful. The person skilled in the art knows how to select appropriate combinations of systems and fluorescent labels and software.

Counting the fluorescent labels (steps D) and d)), e.g. the first and second fluorescent labels, may include image processing, for example convolution, thresholding, dilution and erosion, spot identification and quantification, and the like. Techniques are well known in the art.

In the seventh and eighth method, the selection of the target species in step C1) may be performed according to the minimal detection procedure. Alternatively or additionally, the selection of the target species in step C1) may be performed according to the maximal detection procedure. This means that all of the target species may be selected with the maximal detection procedure or all of the target species may be selected with the minimal detection procedure. Alternatively, some of the target species may be selected with the minimal detection procedure and some of the target species may be selected with the maximal detection procedure. It is also possible to define selection criteria according to which a target species is selected if its expected concentration cᵢ is between minimal detection procedure and maximal detection procedure (see below).

Definitions of the minimal detection procedure and the maximal detection procedure are given in the course of the following explanation of a sub-method for selecting target species for the individual secondary samples and eventually corresponding coverslip regions. In order to make the reading easier, we refer to “channel” as a synonym for “secondary sample and eventually corresponding coverslip region”.

### Sub-Method for Selecting Target Species for Dilution Channels

With a number of target species m to be detected (for a definition of target species see above) and a known reference table of their expected, e.g. typical, mean concentrations cᵢ and the corresponding standard deviations or, as determined by prior reference experiments or publicly available data, that establish the typical concentration of the biological species, and the number molecular species molecules per cell (Kraal et al, Plos One 2014 as cited above, rrnDB as cited above). If standard deviations are not available, an estimated value can be chosen as a safety measure. The integer i counts through the target species, i.e. i runs from 1 to m. For this, the target species are assumed sorted according to their expected, e.g. typical, mean concentration. The highest cᵢ is referred to as cₘₐₓ, and cₘᵢₙ refers to the lowest cᵢ. Accordingly, cₘₐₓ=cₘ and cₘᵢₙ=c₁

The following values are also given, i.e. known:
K number of channels (i.e. secondary samples and eventually the corresponding coverslip regions).
n_{acq} number of images acquired per coverslip region at different positions. This parameter can be freely chosen, an increase improves the dynamic range, but also increases the acquisition time. Its maximum is determined by the size of the coverslip region. Imaging an area of the coverslip region multiple times is possible, but not intended here. If this is performed, care must be taken not to count spots more than once.
Nₘₐₓ maximum number of spots per image for quantification (spot refers to fluorescent punctae in the images acquired for analysis, as used in Woehrstein et al, cited above. See FIG. 3 B, C, and section “Results/Tunable Brightness”; this value is a property that depends on the imaging system and the analysis algorithm. For example, if the analysis algorithm allows for the analysis of spots up to a density of 1 spot/μm, and the imaging system yields images of 10.000 μm², Nₘₐₓ would be 10.000.
fᵢ capture factor: this is a calibration factor that depends on assay parameters like incubation times and the ratio of volume/surface area of the coverslip regions, as well as the accessibility. It is pre-determined in separate experiments.
Nᵢ expected number of spots of species I per image; Nᵢ is calculated from fᵢ and cᵢ: Nᵢ=fᵢ*cᵢ;
α variation safety factor: α is a factor that is used for the selection process in order to account for the possibility that an actual concentration of a target species may differ from the expected value cᵢ; makes sure to detect a target species appropriately if its concentration is between cᵢ−α*σ and cᵢ+α*σ.
r_{dyn} logarithmic dynamic range; ideally r_{dyn}=log(Nₘₐₓ*n_{acq}/m) (assuming equal concentrations of all target species and no unspecific binding-practically, assuming r_{dyn}=log(Nₘₐₓ*n_{acq}/(10*m)) gives good results and is preferred.)
r_{K} logarithmic dynamic range shift between consecutive coverslip regions. This is equal to the logarithm of the dilution factor between the coverslip regions.

Then the total logarithmic dynamic range that shall be measured is

$${r}_{t o t} = log ( ( {c}_{m ax} + a * {σ}_{m a x} ) / ( {c}_{min} - α * {σ}_{min} ) )$$

A dynamic range shift r_{K} from channel to channel may then be
rₖ=rₜₒₜ/K, with a fixed number of channels K.

Alternatively, the dynamic range shift r_{K} can be fixed such that a meaningful overlap is given between consecutive channels, e.g. r_{K}=r_{dyn}/2. In this case, the total number of coverslip regions used depends on the total concentration range to cover (and thus, rₜₒₜ).

The channels can then be ordered using the integer j running from 0 to K−1. The fraction of target species concentrations in channel j to those in channel 0 is then 10\hat{ }(j*r_{K}). Here, j=0 denotes the lowest concentration (highest dilution) channel, and j=K−1 denotes the highest concentration (lowest dilution) channel. The concentration ranges the channels are designed for can be expressed as going from c_{j, lo} to c_{j, hi} (without these necessarily being the limits of detection):

$${c}_{j , lo} = {( {c}_{min} - α * {σ}_{mi n} )}^{*} 1 {0}^{\hat{}} ( j * {r}_{K} )$$

$${c}_{j , hi} = ( {c}_{min} - α * {σ}_{min} ) * {10}^{\hat{}} ( ( j + 1 ) * {r}_{K} )$$

For selecting those target species among the m target species that are to be detected in each channel, some methods stand out amongst others:
a) With the “minimal detection procedure”, target species are detected only to satisfy the variation safety factor (i.e. within a concentration of cᵢ−α+σᵢ to cᵢ+α*σᵢ).
   A target species i is designated to be detected in a channel j if
   c_{j, lo}≤cᵢ+α*σᵢ and
   c_{j, hi}<cᵢ−α*σᵢ,
   wherein j is an integer counting through the channels, i.e. j runs from 0 to K−1.
b) With the “maximal detection procedure”, target species are detected wherever the maximally expected concentration is in the linear range of measurement.
   A target species i is designed to be detected in a channel j if
   1<c_{j, hi}/(cᵢ+α*σᵢ)≤r_{dyn}
   wherein j is again the integer counting through the channels, i.e. j runs from 0 to K−1.
c) With the “singular detection procedure”, each target species is only detected in one channel. The assigned channel can be calculated using:
   j_{assign}=round(rₖ/2+log((cᵢ/(cₘᵢₙ−α*σₘᵢₙ))/rₖ)

With the channels having target species assigned according to the above sub-method or any other suitable method, we can calculate the expected number of spots per image Nⱼ:

$${N}_{j} = {10}^{j \cdot {r}_{k}} {\sum }_{i}^{i i n channel j} {f}_{i} \cdot {c}_{i}$$

If Nⱼ is larger than Nₘₐₓ for any channel, the lowest dilution secondary sample can be derived from the primary sample by diluting by factor d:

d=Nₘₐₓ/maximum(Nⱼ)

Otherwise, d can be chosen to be close to 1 (as close as transferring to hybridization buffer allows). The term hybridization buffer” refers to a buffer promoting hybridization between oligonucleotides, e.g. buffer RX20 or buffer B (recipes see below).

The number of target species assigned to each channel is

$${L}_{j} = {\sum }_{i}^{i i n channel j} 1$$

Alternatively, coverslip region dilutions can be assigned differently, e.g. non-uniformly, and target species can be assigned to coverslip regions using different algorithms, depending on the specific collection of target species to be analyzed.

With these calculations, a procedure according to the invention may be as follows:
Take a stool sample, purify its RNA (or optionally rRNA)
Dilute it by d while transferring it into hybridization buffer (the result may be the primary sample)
Incubate part of this sample, i.e. this is the lowest dilution secondary sample, in channel K−1
Dilute another part of it by 10\hat{ }rₖ (thus creating another secondary sample), and incubate in channel K−2
Repeat until total dilution of d*10\hat{ }(k*rₖ) in channel 0 (channel 0 receives the highest dilution secondary sample)
After incubation, flush out unbound RNA
Incubate channel j with fluorescent labels (e.g. DNA-nanoparticles) for all target species selected for it according to the sub-method above
Flush out unbound fluorescent labels (e.g. DNA-nanoparticles)
Image
Perform image processing if and/or as necessary, e.g. convolution, thresholding, dilution and erosion, spot identification, and the like
Count spots in the images for each species i, multiply by d*10\hat{ }((K−j)*rₖ) and divide by fᵢ to get the measured concentration in the undiluted sample.
If one species was in multiple channels, perform error calculation to provide a precision for the measurement.

As mentioned above, another method according to the invention may be to incubate the secondary dilutions with fluorescent labels in separate reaction containers and immobilize the reacted constructs on coverslip regions as described before, afterwards flushing out any unbound moieties from the coverslip regions.

Particularly, this method may comprise the following steps:
Take a stool sample, purify its RNA (or optionally rRNA)
Dilute it by d while transferring it into hybridization buffer (the result may be the primary sample)
Put part of this sample, i.e. this is the lowest dilution secondary sample, in reaction container K−1, intended for channel K−1
Put fluorescent labels (e.g. DNA-nanoparticles) for all target species selected for channel K−1 according to the sub-method above into reaction container K−1 and incubate
Dilute another part of the primary sample by 10\hat{ }rₖ (thus creating another secondary sample), and put it into reaction container K−2, intended for channel K−2
Put fluorescent labels (e.g. DNA-nanoparticles) for all target species selected for channel K−2 according to the sub-method above into reaction container K−2 and incubate
Repeat until total dilution of d*10\hat{ }(k*rₖ) in reaction container 0, intended for channel 0 (channel 0 receives the highest dilution secondary sample)
After incubation, transfer mixtures from reaction containers into corresponding channels
Incubate channels
Flush out unbound moieties (i.e. RNA and fluorescent labels (e.g. DNA-nanoparticles))
Image
Perform image processing if and/or as necessary, e.g. convolution, thresholding, dilution and erosion, spot identification, and the like
Count spots in the images for each species i, multiply by d*10\hat{ }((K−j)*rₖ) and divide by fᵢ to get the measured concentration in the undiluted sample.
If one species was in multiple channels, perform error calculation to provide a precision for the measurement.

Alternatively and or additionally, the fluorescent labels may be provided to the primary sample under conditions allowing for binding of the fluorescent labels with the corresponding target species, followed by preparation of the secondary samples, optionally immobilization and flushing out any unbound moieties from the coverslip regions.

Particularly, this method may comprise the following steps:
Take a stool sample, purify its RNA (or optionally rRNA)
Dilute it by d while transferring it into hybridization buffer (the result may be the primary sample)
Add fluorescent labels (e.g. DNA-nanoparticles) for all target species and incubate
Incubate part of this sample, i.e. this is the lowest dilution secondary sample, in channel K−1, which is pre-configured to only capture the target species selected according to the sub-method above.
Dilute another part of it by 10\hat{ }rₖ (thus creating another secondary sample), and incubate it in channel K−2, which is pre-configured to only capture the target species selected according to the sub-method above.
Repeat until total dilution of d*10\hat{ }(k*rₖ) in channel 0 (channel 0 receives the highest dilution secondary sample)
Flush out unbound moieties (i.e. RNA and fluorescent labels (e.g. DNA-nanoparticles))
Image
Perform image processing if and/or as necessary, e.g. convolution, thresholding, dilution and erosion, spot identification, and the like
Count spots in the images for each species i, multiply by d*10\hat{ }((K−j)*rₖ) and divide by fᵢ to get the measured concentration in the undiluted sample.
If one species was in multiple channels, perform error calculation to provide a precision for the measurement.

The present invention also relates to a system comprising a fluorescent microscope, at least one sample carrier with at least two coverslip regions, one or more samples to be analyzed and a processor, wherein the system is configured, adapted and/or programmed to, preferably automatically, perform a method according to any one of the preceding claims. The one or more samples to be analyzed may comprise a primary sample and/or one or more secondary samples. The system may comprise software, wherein the system is configured to execute the software, and wherein the software is configured to control the system in such a way as to perform the method according to any one of the preceding claims.

The prior art cited herein is incorporated herein by reference in its entirety.

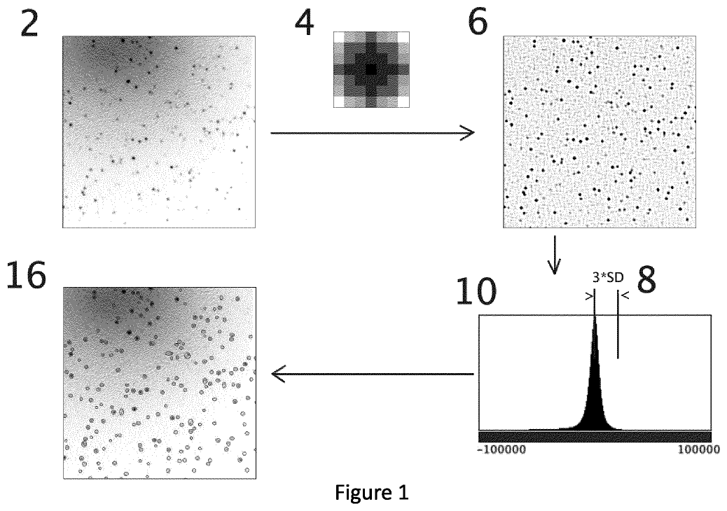

The following describes an exemplary embodiment of the present invention with reference to FIG. 1.

FIG. 1 shows an exemplary analysis procedure. Note that the specific numbers for volumes, masses, sizes and the like are exemplary and may vary in other embodiments. Particularly, they may be scaled up or down to fit other reaction chambers than the one used in the following embodiment, which is “μ-Slide VI 0.1 with a sticky bottom” from ibidi GmbH (alternatively, the “sticky-Slide VI 0.4” may be used; the volumes of the liquids are preferably adapted to the volume of “sticky-Slide VI 0.4”). Of course, other embodiments may comprise other target species and other related molecules, such as the fluorescent labels.

### Sample Collection

1. Collect 1.5 g stool sample in a DNA/RNA Shield Fecal Collection Tube (Zymo Research Europe GmbH, Freiburg, Germany; shortly Zymo or Zymo Research in the following) prefilled with 9 ml of DNA/RNA Shield solution (Zymo).
2. Immediately store samples at −20° C. (keep for up to 1 month).
   RNA Isolation from Stool Sample (Zymobiomics RNA Mini Kit, Zymo Research)
1. Thaw sample on ice and transfer 750 μl of well-mixed sample+DNA/RNA shield (Zymo) to BashingBead Lysis Tubes (Zymo).
2. Secure tube in vortex adapter fitted for 2 ml tubes and vortex at highest speed for 45 min.

The following steps are performed as suggested by the manufacturer:
3. Centrifuge the tube for 1 minute to pellet debris.
4. Transfer up to 200 μl of the cleared supernatant into a nuclease-free tube.
5. Add an equal volume of RNA Lysis Buffer (1:1) (Zymo) to the supernatant and mix well.
6. Add an equal volume of ethanol to the sample and mix.
7. Transfer mixture to a Zymo-spin IIICG column in collection tube and centrifuge. Then, discard flow-through.
8. Add 400 μl RNA prep buffer (Zymo) to the column and centrifuge. Discard flow-through and transfer column into nuclease-free tube (Zymo)
9. Add 400 μl RNA Wash buffer (Zymo) to the column and centrifuge. Discard flow-through and transfer column into nuclease-free tube (e.g. “PCR clean” tubes from Eppendorf GmbH).
10. Add 85 μl DNase/RNase-free water (Zymo) directly to the column matrix (C1006-50-G, Zymo), then centrifuge to elute.
11. DNase I treatment: to the eluate add 10 μl DNA Digestion Buffer (Zymo) and 5 μl DNase I (Zymo) and mix gently by manually inverting top and bottom a few times. Incubate at room temperature for 15 min.
12. Add 2 volumes of RNA Lysis Buffer (Zymo) to the sample (2:1) and mix.
13. Add an equal volume ethanol (95-100%) (1:1) and mix well.
14. Transfer the mixture into a new Zymo-Spin IIICG Column in a collection tube and centrifuge. Discard flow-through.
15. Add 400 μl RNA prep buffer (Zymo) to the column and centrifuge. Discard flow-through.
16. Add 700 μl RNA wash buffer (Zymo) to the column and centrifuge. Discard flow-through.
17. Add 400 μl RNA wash buffer (Zymo) and centrifuge the column for 1 min to ensure complete removal of the wash buffer. Carefully transfer the column into nuclease-free tube.
18. Add 100 μl DNase/RNase-free water to the column matrix and centrifuge to elute.
19. Place a Zymo-Spin III HRC Filter in a new collection Tube and add 600 μl ZymoBiomics Prep Solution. Centrifuge at 8000 g for 3 min.
20. Transfer the eluted RNA (of step 18) into the prepared Zymo-Spin III-HRC Filter in a nuclease-free tube. Then centrifuge exactly 16000 g for 3 min.
21. Collect the resulting filtered RNA as a primary sample.

The result of “RNA isolation from stool sample” may be a primary sample. The transfer into hybridization buffer may be advantageous at this time. The preparation of the primary sample according to this protocol is optionally and exemplary. Other suitable protocols may be used for providing the primary sample.

### Selection of Number and Dilution Factors of Secondary Samples

1. Select target species to be analyzed.
2. Select/determine the number of secondary samples and the dilution factors for the secondary samples according to the selected target species and the expected concentrations cᵢ of the target species. Suitable selection procedures are described herein above.

### Sequence Design for Immobilization Molecules & Fluorescent Label Handles

Herein, the term “barcode” is used as a synonym for fluorescent label. The terms “capture adapter” and “capture strand” and “surface capturing handle” are used as synonyms for “immobilization molecule”. The terms “barcode handle” and “barcode strand” describe the part of the fluorescent label (or barcode) that mediate the binding of target species and fluorescent label.

### Part A—Database Preparation:

1. Install R, version 4.0.4 (https://www.r-project.org/)
2. Install DECIPHER package and load it with R (https://www.bioconductor.org/packages/release/bioc/html/DECIPHER.html)
3. Download SILVA database in the “fasta” file format (https://www.arb-silva.de/browser/, click on SILVA to add to cart, and “download as fasta file with gaps”) and read into R. Gut bacteria from three publications were collected and used to generate a shortened reference database by extracting relevant species.
4. Use the DECIPHER function AlignSeqs( ) to align the reference database into equally sized sequences and find and complete multiple partial sequences (for details, see ES Wright (2015) “DECIPHER: harnessing local sequence context to improve protein multiple sequence alignment”. BMC Bioinformatics, doi:10.1186/s12859-015-0749-z).
5. Use the “DesignProbe” function implemented in the DECIPHER package for designing possible sequences (Parameter settings: minLength=23, maxLength=23, hybTemp=30, P=2.5e-11, FA=15, numProbeSets=50, minCoverage=0.9, minGroupCoverage=0.2, batchSize=1000, target=“SSU”)

### Part B: Target Species Analysis:

1. Download your target species sequence of interest from SILVA database (https://www.arb-silva.de/no_cache/download/archive/release_138.1/Exports/).
2. Insert sequence to RNA fold WebServer (http://rna.tbi.univie.ac.at/cgi-bin/RNAWebSuite/RNAfold.cgi) to determine predicted secondary structure and “degree of accessibility” of individual nucleotides.
3. Extract RNAfold outputs:
   a. Download Results of thermodynamic ensemble prediction as Ct format, and open in Microsoft Excel.
   b. Click “View in FORNA” under “Centroid structure drawing encoding base-pair probabilities”, click “Colors/Custom/Set”, and add the base-pair probability values to a column in Excel.
4. Merge RNAfold output with the designed sequences from Part A (step 4; R; package plyr, join function)
5. Choose sequence combinations with the best score.

This procedure results in 23 nt long sequences that can be used as handles (i.e. single-stranded oligos configured to specifically bind a target, and attached to something, e.g. DNA-Nanostructures or a surface) for the DNA-Nanostructures in order for them to act as the aforementioned fluorescent labels targeting molecular target species. Additionally, 23 nt long sequences are generated which can be used for the capture adapter by ordering one oligonucleotide comprising this 23nt long sequence and the complementary sequence of the surface strand mentioned below. For a generic 16S surface capturing instead of species-specific capturing, one or multiple conserved regions, as for example Seq-ID No 2 can be used.

In the following procedure, a biotinylated “surface strand” with a generic sequence (Seq-ID No 3: “gaatcggtca cagtacaacc g”, 5′ biotin-modification) and a “capture adapter” that comprises the complementary sequence of the surface strand as well as the sequence resulting from this current analysis are used. For generic capture, the resulting capture adapter may for example be Seq-ID No 4.

### Fold DNA-Nanostructures:

Using the barcode handle sequence derived above, fold DNA-nanostructures as described in J B Woehrstein et al., Science Adv (2017) https://doi.org/10.1126/sciadv.1602128. The number and types of DNA-nanostructures, particularly their fluorescent dyes, i.e. colors, needs to be adapted to the number of target species, to the number of secondary samples and to the dilution factors of the secondary samples as described herein.

The resulting DNA-Nanostructures can be used as fluorescent labels targeting molecular

species, i.e. target species.

### Hybridization Protocol:

### Preparation of a Fluid Manipulation Device/Flow Chamber:

3. Incubate a cover slip in Isopropanol for 5 min and dry under a N₂ stream.
4. Immediately after that, place a “μ-Slide VI 0.1 with a sticky bottom” (ibidi GmbH) onto the cover slip, bonding them together via the sticky bottom, and thus creating flow channels as coverslip regions.
5. Apply light pressure to the cover slip, e.g. using a pipette tip, to improve adhesion between the cover slip and the sticky bottom.

The fluid manipulation device/flow chamber is now ready for use. It is also referred to as “slide”. The fluid manipulation device/flow chamber comprises several, for example six, coverslip regions.

The preparation of the flow chamber is optional and exemplary. Other flow chambers and other preparation procedures are contemplated. It is also possible to provide a suitable ready-for-use flow chamber.

Passivation of Coverslip Regions Against Unspecific Binding & Covering of Coverslip Regions with Immobilization Molecules by Means of Biotin-BSA-Streptavidin Surface (5 Min Incubation):
1. Equilibrate all flow channels (shortly: channels), that μ-Slide VI 0.1 from ibidi-GmbH provides for sample application (i.e. reaction compartments), by adding Buffer A+ to the flow chamber inlet and outlet, then tilt the slide back and forth until a smooth flow is achieved.
2. Prepare biotin-BSA with 1 mg/ml using Buffer A+(definition see below). Prepare 200 μl biotin-BSA per slide (25 L per flow channel).
3. Remove liquid from flow channels.
4. Apply 25 μl of biotin-BSA to each flow channel. Tilt the slide back to ensure even distribution.
5. Seal flow channels with Parafilm and incubate at RT for 5 min.
6. Remove liquid and wash each flow channel 3× with 25 μl of Buffer A+. Do not remove final wash until ready to apply Streptavidin (see next step).

7. Prepare Streptavidin with 100 μg/ml using Buffer A+. Prepare 200 μL Streptavidin per slide (25 μL per flow channel).
8. Remove liquid from flow channels.

9. Apply 25 μl of Streptavidin to each flow channel. Tilt the slide back to ensure even distribution.
10. Seal flow channels with Parafilm and incubate at RT for 5 min.
11. Remove liquid and wash each flow channel 3× with 25 μl of Buffer A+. Do not remove final wash until ready to apply biotinylated Surface Strand (see next steps).

Buffer A+ ingredients:
10 mM Tris-HCl with pH 7.5
100 mM NaCl
0.05% Tween 20

Buffer B+ ingredients:
10 mM Tris-HCl with pH 8.0
10 mM MgCl2
1 mM EDTA

The result is a flow chamber with six flow channels with a streptavidin-modified surface.

### Biotinylated Surface-Strand (5 Min Incubation)

1. Prepare biotinylated Surface Strand with 1 μM in Buffer A+.
2. Prepare 200 μL biotinylated Surface Strand per slide (25 μL per flow channel).
3. Remove liquid from flow channels.
4. Apply 25 μl Surface-Strand to each flow channel. Tilt the slide back to ensure even distribution.
5. Seal flow channels with Parafilm (Bemis Company Inc.) or similar sealant and incubate at room temperature (RT) for 5 min.
6. Remove liquid and wash each flow channel 3× with 25 μl of Buffer A+. Do not remove final wash until ready to apply Capture-Adapter Strand.

The result of this procedure is a flow chamber with six flow channels with a surface comprising single DNA oligos comprising the sequence Seq-ID No 3.

### Capture-Adapter Strand (5 Min Incubation)

For each coverslip region, prepare a capture-adapter mix comprising those capture adapters resulting from the step “Sequence Design, Part B” that target the target species selected according to a sub-method mentioned herein above for the respective coverslip region.

For all channels j, prepare capture-adapter mix:
1. Dilute all capture adapters to 200 μM in Buffer A+
2. For channel j, mix capture adapters selected for slide j.
3. Dilute in Buffer A+ to a final concentration of each capture adapter of 1 μM.

Incubate the capture adapter mixes to the respective coverslip regions:
4. Remove liquid from channels.
5. Apply 25 μl of the corresponding capture-adapter mix to each channel. Tilt the slide back to ensure even distribution.
6. Seal channels with Parafilm and incubate at 37° C. for 5 min.
7. Remove liquid and wash each channel 3× with 25 μl of Buffer A+. Do not remove final wash until ready to apply RNA Solution.

The result of this procedure is a flow chamber with six flow channels with a surface comprising single DNA oligos comprising the capture sequences generated in the step “Part B: Target Species Analysis”. These are complementary to the target species (e.g. 16S rRNA) and can specifically bind them via hybridization. If the approach of capturing all 16S rRNA is chosen, and not capturing different target species in different coverslip regions, the result may be a flow chamber with six flow channels with a surface comprising single-stranded DNA comprising sequence Seq-ID No 2.

### RNA Capture (45 Min Incubation)

1. From the primary sample, resulting from the step “RNA isolation from stool sample”, prepare 200 μl 16S rRNA in hybridization solution (here: RX20 with Formamide) per slide (i.e. flow chamber) according to the following table to yield secondary sample K−1. The following recipe assumes an RNA concentration of 1 nM (total RNA of all target species), this is a value that typically yields good results, but it can be adjusted using the dilution factor d.

| Secondary sample K-1 | Volume (μl) | Final concentration |
|---|---|---|
| RNA | 1 | 5 pM |
| H20 | 69 | |
| Formamide | 30 | 15% |
| 2× RX20 (recipe see below) | 100 | 1× |
| TOTAL | 200 | |

2. Prepare dilution solution

| Dilution solution | Volume (μl) | Final concentration |
|---|---|---|
| H20 | 70 | |
| Formamide | 30 | 15% |
| 2× RX20 (recipe see below) | 100 | 1× |
| TOTAL | 200 | |

3. For each of the other secondary samples j, dilute secondary sample j+1 in dilution solution by a factor 10\hat{ }rₖ, as described in a sub-method detailed herein above.
4. Remove wash buffer from flow channels
5. Apply 25 μl of secondary sample to the corresponding channel. Tilt the slide back to ensure even distribution.
6. Seal flow channel with Parafilm and incubate at 37° C. for 45 min
7. Remove liquid and wash each flow channel 3× with 25 μl of Buffer A+. Do not remove final wash until ready to apply Barcode Solution.

RX20-Buffer ingredients:
4×SSC (saline sodium citrate-Buffer, consisting of aqueous solution of 150 mM potassium chloride and 15 mM tri potassium citrate at pH 7.0)
5% Dextran Sulfate
0.1% Tween20
5×Denhardt's Medium

### Barcode Hybridization (80 Min Incubation)

In this step, fluorescent labels that are configured to directly or indirectly bind specifically to the target species are provided to the secondary samples under conditions allowing for labeling the target species.

For each coverslip region, prepare a Barcode solution comprising those barcodes resulting from the step “Fold DNA-Nanostructures” that target the target species selected according to a sub-method mentioned herein above for the respective coverslip region. If the number of target species per coverslip region Lⱼ does not exceed 25, dilute the barcodes to an input concentration of approximately 2.5 nM each, and use the following table as a preparation recipe. If Lⱼ exceeds 25, adjust barcode pre-dilution and water addition accordingly.

| Barcode Solution for | | |
|---|---|---|
| channel j | Volume (μl) | Final concentration |
| Barcodes 2.5 nM (per target | 1.6 | 50 pM |
| species), total Lⱼ barcodes | | |
| types | | |
| H20 | 38.4 (for L = 1); | |
| | 40 − 1.6*L | |
| 2× RX20 | 40 | 1× |
| TOTAL | 80 | |

1. Prepare 80 μl Barcode Solution per coverslip region
2. Remove wash buffer from flow channels
3. Apply 12 μl of Barcode Solution to the respective flow channel (coverslip region). Tilt the slide back to ensure even distribution
4. Incubate at 37° C. for 80 min (1 hour 20 min)
5. Wash each flow channel 3× with 25 μl of Buffer B+. Do not remove final wash.
6. Seal the flow channel tightly with Parafilm or clear tape.
7. Proceed to imaging

### Imaging

1. Place sample, i.e. flow chamber, onto a microscope, here a Zeiss AxioObserver 7 with 60×/1.4 objective, Colibri 7 illumination, AxioCam 506.
2. Acquire z-stacks (7 planes at 300 nm spacing) at multiple (n_{acq}) positions and the color channels corresponding to the dyes used in “Fold DNA-Nanostructures”
3. Use Zeiss ZEN software postprocessing module “Extended depth of focus” to project the z planes.
   Processing Images with ImageJ (Fiji) and COMDET V.0.5.4 Plugin, Counting the Fluorescent Labels

The description of image processing is given for ImageJ (Fiji) and OMDET V.0.5.4 Plugin. However, any suitable image processing may be used.
1. Load images from the Zeiss Observer microscope with czi extension directly into Fiji. (Nature methods 9(7): 676-682, PMID 22743772)
2. Adjust contrast to see the fluorescent spots to check the acquisition was performed correctly.
3. Load the COMDET plugin, press “Detect particles”, adjust parameters in the pop-up window according to your needs (“preview calculation button” must be checked) https://github.com/ekatrukha/ComDet

### Software Steps:

The original image 2 is convoluted with a Gaussian Mexican hat filter 4 (user defines the size of the Mexican hat, here four pixels). The result is a convoluted image 6. A histogram 10 of the convoluted image 6 is made via automatic threshold detection and fitted with a Gaussian distribution to find a threshold 8 for pixels representing a spot (e.g. 3*SD, with SD being the standard deviation of the fitted gaussian distribution). The convoluted image 6 is thresholded with this threshold 8 and the result is a thresholded mask. Some speckles are removed with dilution/erosion operations (for example as described in the open source code Katrukha E. 2020, ComDet plugin for ImageJ, v0.5.3, Zenodo, doi:10.5281/zenodo.4281064), resulting in a filtered mask. Leftover spots that have not been removed in the previous step are considered to be valid, and consist of a number of contiguous pixels. They are used to calculate “centroid x,y” and the resulting marks (i.e. centroids) are overlayed on the original image 2, resulting in the final image 16. Also, the pixels making up these leftover spots are used for calculation of Integrated Intensity (IntegratedInt) and NArea values.

The values of IntegratedInt are copied into a table, combining the values for the different color channels acquired, for each spot. This table is used to assign barcodes and thus the target species they were designed for, according to the methods described in Woehrstein et al, cited above. Finally, the numbers of detected target species molecules N_{i,det} are converted into detected concentrations tᵢ of the target species using the pre-determined parameter fᵢ: tᵢ=N_{i,det}/fᵢ

If the concentration of the biological species is of interest, they can be estimated using the molecular species concentrations in combination with the aforementioned calibration measurements or database entries such as in rrnDB (cited above).

### Seq-IDs

<210> 1
<211> 22
<212> DNA
<213> Artificial Sequence
<220>
<223> 16S conserved region
<400> 1
AAACTCAAAGGAATTGACGGGG 22
<210> 2
<211> 22
<212> DNA
<213> Artificial Sequence
<220>
<223> 16S primer
<400> 2
CCCCGTCAATTCCTTTGAGTTT
22
<210> 3
<211> 21
<212> DNA
<213> Artificial Sequence
<220>
<223> Surface Strand, 5′ modification: biotin
<400> 3
gaatcggtca cagtacaacc g 21
<210> 4
<211> 45
<212> DNA
<213> Artificial Sequence
<220>
<223> capture adapter
<400> 4
CCCCGTCAATTCCTTTGAGTTTtttggttgtactg tgaccgattc 45

1. Method for performing a microbiome-analysis, comprising the steps:
a) providing a primary sample derived from a microbiomic sample, the primary sample comprising a first target species at a first concentration and a second target species at a second concentration and different from the first target species,
wherein the first target species is indicative of a first element of a microbiome of the microbiomic sample and the second target species is indicative of a second element of the microbiome, and
wherein the second concentration is higher than the first concentration;
b) providing two or more secondary samples derived from the primary sample, the two or more secondary samples including at least a highest dilution secondary sample and a lowest dilution secondary sample,
wherein the at least two secondary samples have different dilutions, and wherein the lowest dilution secondary sample may be diluted or undiluted is the primary sample or a dilution of the primary sample;
c) providing a first fluorescent label that is configured to directly or indirectly bind specifically to the first target species to the lowest dilution secondary sample under conditions allowing for labeling the first target species with the first fluorescent label, and providing a second fluorescent label that is configured to directly or indirectly bind specifically to the second target species to the highest dilution secondary sample, but not to the lowest dilution secondary sample, under conditions allowing for labeling the second target species with the second fluorescent label;
d) analyzing the first and second target species according to the steps:
d1) imaging the secondary samples with fluorescence microscopy and counting the first and second fluorescent labels in the corresponding secondary samples;
d2) calculating the ratio of the first target species to the second target species from the results of step d1) and the dilutions of the corresponding secondary samples.

2. The method according to claim 1, wherein step c) comprises the steps:
c1) applying the secondary samples to corresponding coverslip regions, wherein the coverslip regions are modified according to the corresponding secondary sample and in the following way:
c1.1) each coverslip region is passivated against unspecific binding of the first and second target species and against unspecific binding of the fluorescent labels;
c1.2) each coverslip region is covered with immobilization molecules bound, directly or indirectly, to the coverslip, wherein the immobilization molecules are configured to, directly or indirectly, bind the first and the second target species;
c2) removing unbound individuals of the first and second target species from the coverslip regions;
c3) providing the first fluorescent label to the lowest dilution secondary sample under conditions allowing for labeling the first target species with the first fluorescent label, and providing the second fluorescent label to the highest dilution secondary sample but not to the lowest dilution secondary sample, under conditions allowing for labeling the second target species with the second fluorescent label
c4) removing unbound individuals of the first and second fluorescent labels from the coverslip regions.

3. Method for performing a microbiome-analysis, comprising the steps:
a) providing a primary sample derived from a microbiomic sample, the primary sample comprising a first target species at a first concentration and a second target species at a second concentration and different from the first target species,
wherein the first target species is indicative of a first element of a microbiome of the microbiomic sample and the second target species is indicative of a second element of the microbiome, and
wherein the second concentration is higher than the first concentration;
b) providing two or more secondary samples derived from the primary sample, the two or more secondary samples including at least a highest dilution secondary sample and a lowest dilution secondary sample,
wherein the at least two secondary samples have a different dilution, and wherein the lowest dilution secondary sample is the primary sample or a dilution of the primary sample;
c) providing a first fluorescent label that directly or indirectly binds specifically to the first target species to the lowest dilution secondary sample under conditions allowing for labeling the first target species with the first fluorescent label and providing a second fluorescent label that directly or indirectly binds specifically to the second target species to the highest dilution secondary sample under
conditions allowing for labeling the second target species with the second fluorescent label; and
wherein in the lowest dilution secondary sample the first target species is being immobilized for analysis and the second target species is not being immobilized for analysis,
wherein in the highest dilution secondary sample the second target is immobilized for analysis and the first target is not immobilized for analysis;
d) analyzing the first and second target species according to the steps:
d1) imaging the secondary samples with fluorescence microscopy and counting the first and second fluorescent labels in each secondary sample; d2) calculating the ratio of the first target species to the second target species from the results of step d1) and the dilutions of the corresponding secondary samples.

4. The method according to claim 3, wherein step c) comprises the steps:
c1) applying the secondary samples to corresponding coverslip regions, wherein the coverslip regions are modified according to the corresponding secondary sample and in the following way:
c1.1) each coverslip region is passivated against unspecific binding of the first and second target species and against unspecific binding of the fluorescent labels;
c1.2) the coverslip region corresponding to the lowest dilution secondary sample is covered with first immobilization molecules bound, directly or indirectly, to the coverslip, wherein the first immobilization molecules are configured to, directly or indirectly, specifically bind the first target species;
c1.3) the coverslip region corresponding to the highest dilution secondary sample is covered with the second immobilization molecules bound, directly or indirectly, to the coverslip, wherein the second immobilization molecules are configured to, directly or indirectly, specifically bind the second target species;
c2) removing unbound individuals of the first and second target species from the coverslip regions;
c3) providing the first fluorescent label to the lowest dilution secondary sample under conditions allowing for labeling the first target species with the first fluorescent label, and providing the second fluorescent label to the highest dilution secondary sample under conditions allowing for labeling the second target species with the second fluorescent label and optionally not providing the second fluorescent label to the lowest dilution secondary sample under conditions allowing for labeling the second target species with the second fluorescent label
c4) removing unbound individuals of the first and second fluorescent labels from the coverslip regions.

5. The method according to claim 4, wherein the two or more secondary samples further include one or more intermediate dilution secondary samples, the method further comprising the step of predetermining a first threshold dilution factor, and wherein step c1) comprises the following condition for modifying the coverslip regions:
c1.4) the coverslip regions corresponding to the intermediate dilution secondary samples are covered with one or both of the first and second immobilization molecules bound, directly or indirectly, to the coverslip, wherein the coverslip regions corresponding to the secondary samples having a dilution factor greater than the first threshold dilution factor are not covered with the first immobilization molecules.

6. The method according to claim 5, comprising the step of predetermining a second threshold dilution factor, and wherein step c1) comprises the following condition for modifying the coverslip regions:
c1.5) the coverslip regions corresponding to the intermediate dilution secondary samples are covered with one or both of the first and second immobilization molecules bound, directly or indirectly, to the coverslip, wherein the coverslip regions corresponding to the secondary samples having a dilution factor equal to or smaller than the second threshold dilution factor are not covered with the second immobilization molecules.

7. The method according to claim 6, comprising the step of predetermining a third threshold dilution factor, and wherein the intermediate dilution secondary samples having a dilution factor greater than the third threshold dilution factor are provided with the second fluorescent label under conditions allowing for labeling the second target species with the second fluorescent label and the intermediate dilution secondary samples having a dilution factor equal to or lower than the third threshold dilution factor are not provided with the second fluorescent label under conditions allowing for labeling the second target species with the second fluorescent label.

8. The method according to claim 7, comprising the step of predetermining a fourth threshold dilution factor, and wherein the intermediate dilution secondary samples having a dilution factor equal to or lower than the fourth threshold dilution factor are provided with the first fluorescent label under conditions allowing for labeling the first target species with the first fluorescent label the intermediate dilution secondary samples having a dilution factor greater than the third threshold dilution factor are not provided with the first fluorescent label under conditions allowing for labeling the first target species with the first fluorescent label.

9. The method according to claim 8, wherein a combination of the first, the second, the third and the fourth threshold dilution factors are equal.

10. The method according to claim 3, wherein the two or more secondary samples further include one or more intermediate dilution secondary samples, wherein one or both of the number and the dilutions of the intermediate dilution secondary samples is adapted to one or both of first and second concentrations and to the ratio of the first and second concentrations.

11.-14. (canceled)

15. The method according to claim 3, wherein one or both of the first fluorescent label and the second fluorescent label comprises at least one DNA-nanostructure with at least one fluorescent dye.

16. The method according to claim 3, wherein the first target species comprises a first nucleic acid, the first nucleic acid comprising a sequence portion S1 specific for the first nucleic acid, and wherein the first fluorescent label comprises a sequence portion S3 at least partially complementary to the sequence portion S1; and
wherein the second target species comprises a second nucleic acid, the second nucleic acid comprising a sequence portion S2 specific for the second nucleic acid, and wherein the second fluorescent label comprises a sequence portion S4 at least partially complementary to the sequence portion S2.

17. (canceled)

18. (canceled)

19. The method according to claim 1,
wherein the first target species comprises a first nucleic acid, the first nucleic acid comprising a sequence portion S1 specific for the first nucleic acid, and wherein the first fluorescent label comprises a sequence portion S3 at least partially complementary to the sequence portion S1;
wherein the second target species comprises a second nucleic acid, the second nucleic acid comprising a sequence portion S2 specific for the second nucleic acid, and wherein the second fluorescent label comprises a sequence portion S4 at least partially complementary to the sequence portion S2;
wherein the immobilization molecules comprise oligonucleotides and the specific binding to corresponding target species occurs via hybridization of at least partially complementary sequence portions on the oligonucleotides and the corresponding target species;
wherein the immobilization molecules comprise one type of oligonucleotides that is configured to bind both the first and the second target species.

20. The method according to claim 16, wherein the immobilization molecules comprise oligonucleotides and the specific binding to corresponding target species occurs via hybridization of at least partially complementary sequence portions on the oligonucleotides and the corresponding target species, wherein the immobilization molecules comprise a first oligonucleotide that is configured to bind the first target species but not the second target species and a second oligonucleotide that is configured to bind the second target species but not the first target species.

21. The method according to claim 3, wherein the second fluorescent label when bound to the second target species is distinguishable in fluorescence microscopy from the first label when bound to the first target species.

22. Method for performing a microbiome-analysis, comprising the steps:
A) providing a primary sample derived from a microbiomic sample, the primary sample comprising m target species at respective concentrations, wherein each of the m target species is indicative of an element of a microbiome of the microbiomic sample;
B) providing u secondary samples derived from the primary sample, the u secondary samples including at least a highest dilution secondary sample and a lowest dilution secondary sample,
wherein the u secondary samples have different dilutions, and
wherein the lowest dilution secondary sample is the primary sample or a dilution of the primary sample;
C) selecting for each secondary sample
C1) those one or more target species among the m target species that are to be prepared for analysis by providing corresponding fluorescent labels that are configured to directly or indirectly bind specifically to the target species under conditions allowing for labeling the one or more target species with the respective fluorescent labels, wherein the fluorescent labels corresponding to different target species are distinguishable in fluorescence microscopy, and
C2) the corresponding fluorescent labels that are configured to directly or indirectly bind specifically to the one or more target species selected in step C1) under conditions allowing for labeling the one or more target species with the respective fluorescent labels;
D) providing the selected fluorescent labels to the corresponding secondary samples as selected in step C) under conditions allowing for labeling the respective target species with the selected fluorescent labels;
E) analyzing the m target species according to the steps:
E1) imaging the secondary samples with fluorescence microscopy and counting the fluorescent labels in the corresponding secondary samples;
E2) calculating the ratios of the m target species from the results of step E1) and the dilutions of the corresponding secondary samples.

23. Method for performing a microbiome-analysis, comprising the steps:
A) providing a primary sample derived from a microbiomic sample, the primary sample comprising m target species at respective concentrations, wherein each of the m target species is indicative of an element of a microbiome of the microbiomic sample;
B) providing u secondary samples derived from the primary sample, the u secondary samples including at least a highest dilution secondary sample and a lowest dilution secondary sample,
wherein the u secondary samples have different dilutions, and
wherein the lowest dilution secondary sample is the primary sample or a dilution of the primary sample;
C) selecting for each secondary sample
C1) those one or more target species among the m target species that are to be prepared for analysis by immobilization, and
C2) corresponding fluorescent labels that are configured to directly or indirectly bind specifically to the one or more target species selected in step C1) under conditions allowing for labeling the one or more target species with the respective fluorescent labels, wherein the fluorescent labels corresponding to different target species are distinguishable in fluorescence microscopy;
D) preparing the secondary samples by
D1) immobilizing the target species according to the selection in step C1)
D2) providing the selected fluorescent labels to the corresponding secondary samples as selected in step C) under conditions allowing for labeling the respective target species with the selected fluorescent labels;
E) analyzing the m target species according to the steps:
E1) imaging the secondary samples with fluorescence microscopy and counting the fluorescent labels in the corresponding secondary samples;
E2) calculating the ratios of the m target species from the results of step E1) and the dilutions of the corresponding secondary samples.

24. The method according to claim 22, wherein
the selection of the target species in step C1) is performed according to one or both of the minimal detection procedure and the maximal detection procedure.

25. A system comprising
a fluorescent microscope
at least one sample carrier with at least two coverslip regions
one or more samples to be analyzed
a processor
wherein the system is configured to perform a method according to claim 1.

26. A system comprising
a fluorescent microscope
at least one sample carrier with at least two coverslip regions
one or more samples to be analyzed
a processor
wherein the system is configured to perform a method according to claim 3.

27. The method according to claim 23, wherein
the selection of the target species in step C1) is performed according to one or both of the minimal detection procedure and the maximal detection procedure.
